(19) United States
(12) Patent Application Publication
(10) Pub. No.: US 2025/0324206 A1
(43) Pub. Date: Oct. 16, 2025

(54) Apparatus, Methods and Computer Programs for Otoacoustic Emission Measurement

(71) Applicant: Nokia Technologies Oy, Espoo (FI)

(72) Inventors: Khaldoon AL-NAIMI, Cambridge (GB); Irtaza SHAHID, Cambridge (GB); Alessandro MONTANARI, Cambridge (GB)

(21) Appl. No.: 19/174,130

(22) Filed: Apr. 9, 2025

(30) Foreign Application Priority Data
Apr. 12, 2024 (GB) 2405192.2

(51) Int. Cl. H04R 25/00

(52) U.S. Cl. CPC H04R 25/30

(57) ABSTRACT

According to various, but not necessarily all, examples there is provided an apparatus comprising: means for causing alternating output of a first audio content and a second audio content, the first audio content comprising a first frequency and the second audio content comprising a different second frequency; and means for receiving, responsive to output of the first audio content and the second audio content, an audio signal indicative of otoacoustic emissions.

### TECHNOLOGICAL FIELD

Examples of the disclosure relate to apparatus, methods and computer programs for otoacoustic emission measurement. Some relate to apparatus, methods and computer programs for distortion product otoacoustic emission measurements using a single loudspeaker.

### BACKGROUND

Otoacoustic emissions (OAEs) are weak acoustic signals that are emitted from within the inner ear, from hairs of the cochlear such as the outer hair cells. The OAEs can be emitted spontaneously or in response to sound stimulation. OAEs can be measured to evaluate the hearing level of a subject.

Distortion Product Otoacoustic Emission (DPOAE) is a widely used method. Conventionally, in DPOAE the cochlea is stimulated simultaneously by two pure tone frequencies, which causes OAEs to be generated by the cochlear hairs at a different frequency. The OAEs are measured and can be used to evaluate hearing levels, including detecting hearing loss at specific frequencies.

### BRIEF SUMMARY

According to various, but not necessarily all, examples there is provided an apparatus comprising: means for causing alternating output of a first audio content and a second audio content, the first audio content comprising a first frequency and the second audio content comprising a different second frequency; and means for receiving, responsive to output of the first audio content and the second audio content, an audio signal indicative of otoacoustic emissions.

In some but not necessarily all examples, the output of the first audio content and the output of the second audio content are non-overlapping temporally.

The first audio content may be a first pure tone and the second audio content may be a second pure tone. The second frequency may be 1.15 to 1.3 times the first frequency.

Causing alternating output of the first audio content and the second audio content may comprise alternating output of the first audio content and the second audio content with gaps between output of the first audio content and output of the second audio content of less than 100 ms.

Receiving an audio signal indicative of otoacoustic emissions may comprise receiving a microphone input indicative of distortion product otoacoustic emissions.

Causing alternating output of the first audio content and the second audio content may comprise, causing output of the first audio content and the second audio content by a single loudspeaker to enable otoacoustic emissions within a subject's ear to be measured.

Causing alternating output of the first audio content and the second audio content may comprise causing output of the first audio content and the second audio content to enable otoacoustic emissions within a subject's ear to be measured. The apparatus may further comprise means for determining, based at least on the received audio signal, the health of the subject's ear.

The apparatus may further comprise means for determining, based at least on the received audio signal, a configuration for outputting further audio content to the subject's ear. The apparatus may further comprise means for causing output of the further audio content based at least in part on the configuration.

According to various, but not necessarily all, examples there is provided a method comprising, causing alternating output of a first audio content and a second audio content, the first audio content comprising a first frequency and the second audio content comprising a different second frequency; and receiving, responsive to output of the first audio content and the second audio content, an audio signal indicative of otoacoustic emissions.

According to various, but not necessarily all, examples there is provided a computer program comprising program instructions, which when executed by an apparatus cause the apparatus to perform at least the following: causing alternating output of a first audio content and a second audio content, the first audio content comprising a first frequency and the second audio content comprising a different second frequency; and receiving, responsive to output of the first audio content and the second audio content, an audio signal indicative of otoacoustic emissions.

According to various, but not necessarily all, embodiments there is provided an apparatus comprising
at least one processor; and
at least one memory including computer program code;
the at least one memory storing instructions that, when executed by the at least one processor, cause the apparatus to perform at least a part of one or more methods described herein.

According to various, but not necessarily all, embodiments there is provided an apparatus comprising means for performing at least part of one or more methods described herein. The description of a function and/or action should additionally be considered to also disclose any means suitable for performing that function and/or action. Functions and/or actions described herein can be performed in any suitable way using any suitable method.

According to various, but not necessarily all, embodiments there is provided examples as claimed in the appended claims.

While the above examples of the disclosure and optional features are described separately, it is to be understood that their provision in all possible combinations and permutations is contained within the disclosure. It is to be understood that various examples of the disclosure can comprise any or all the features described in respect of other examples of the disclosure, and vice versa. Also, it is to be appreciated that any one or more or all the features, in any combination, may be implemented by/comprised in/performable by an apparatus, a method, and/or computer program instructions as desired, and as appropriate. The description of a function should additionally be considered to also disclose any means suitable for performing that function

### BRIEF DESCRIPTION

Some examples will now be described with reference to the accompanying drawings in which:

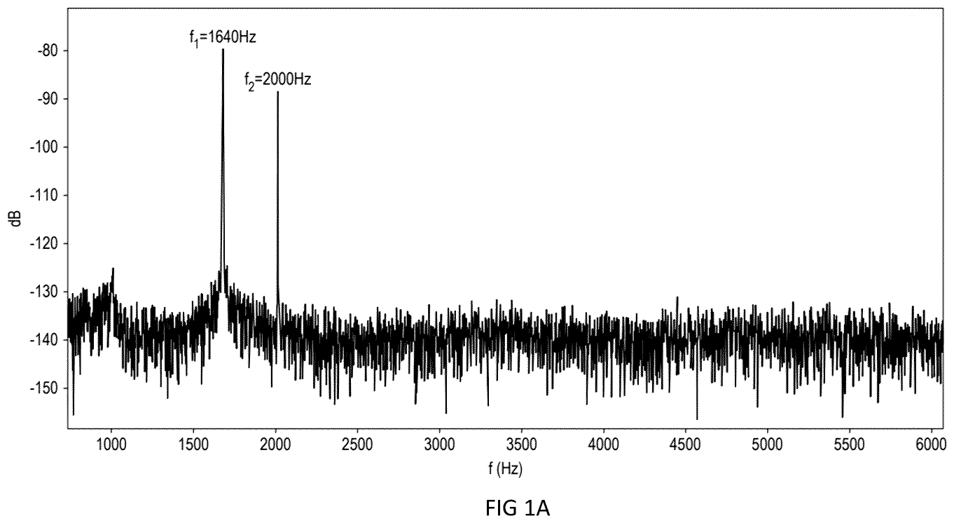

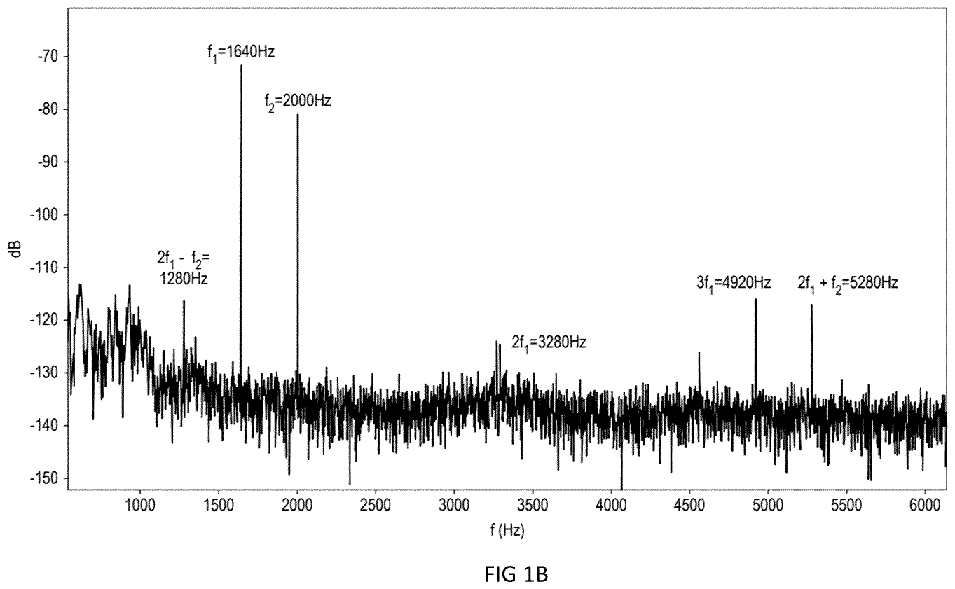

FIGS. 1A and 1B show an example of the subject matter described herein;

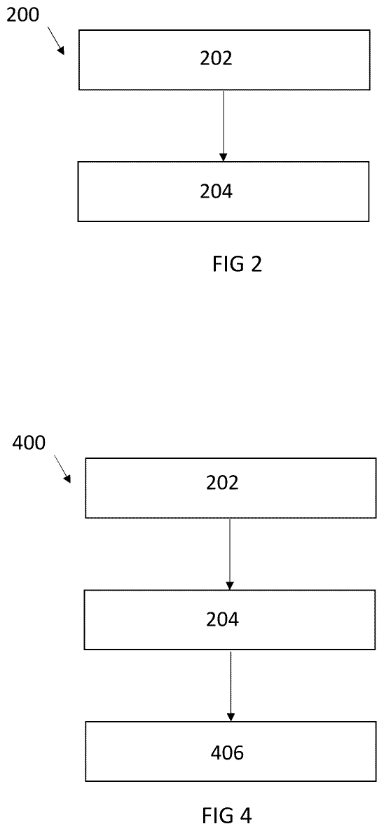

FIG. 2 shows another example of the subject matter described herein;

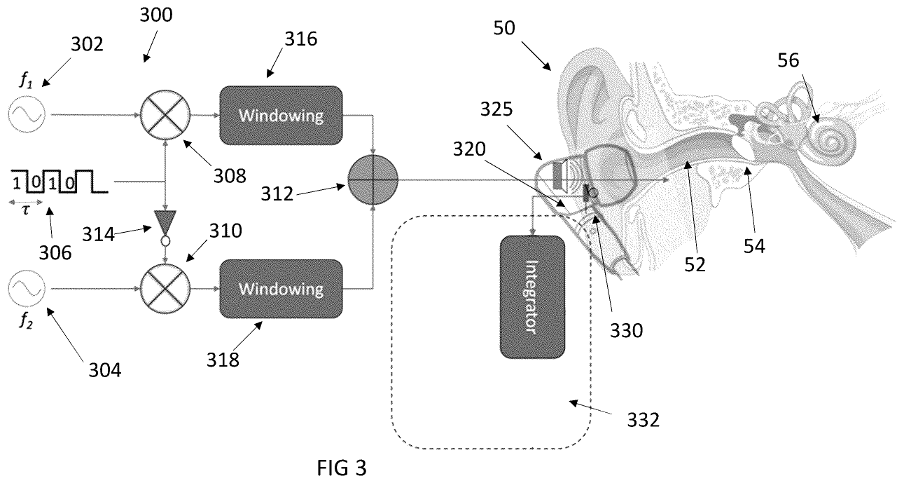

FIG. 3 shows another example of the subject matter described herein;

FIG. 4 shows another example of the subject matter described herein;

FIG. 5 shows another example of the subject matter described herein;

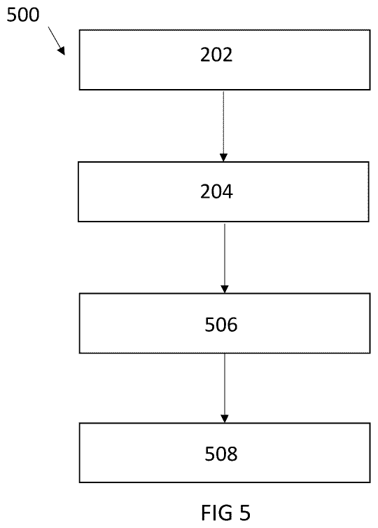

FIG. 6 shows another example of the subject matter described herein;

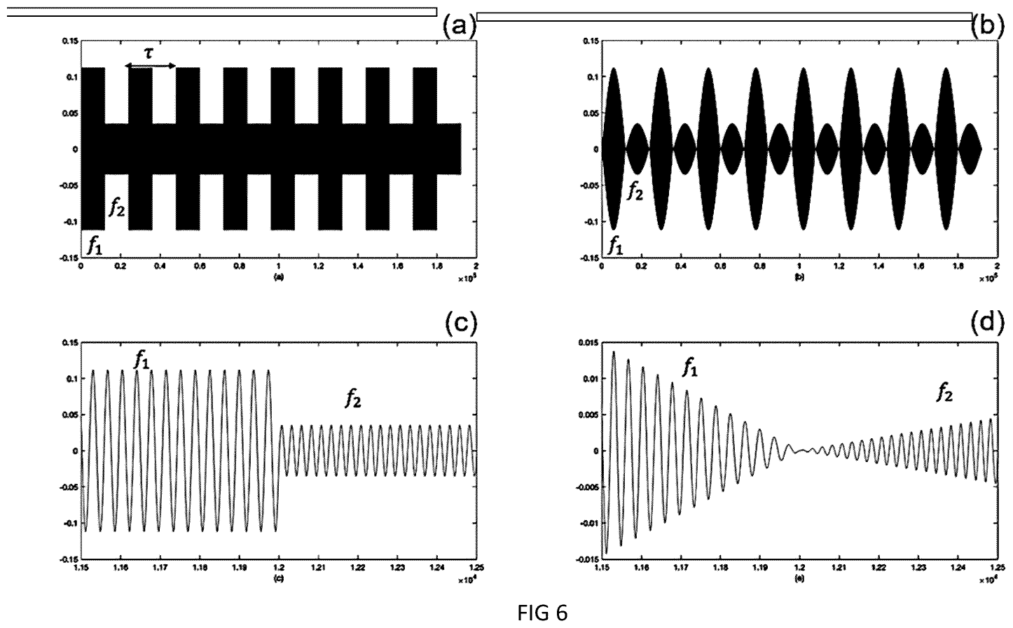

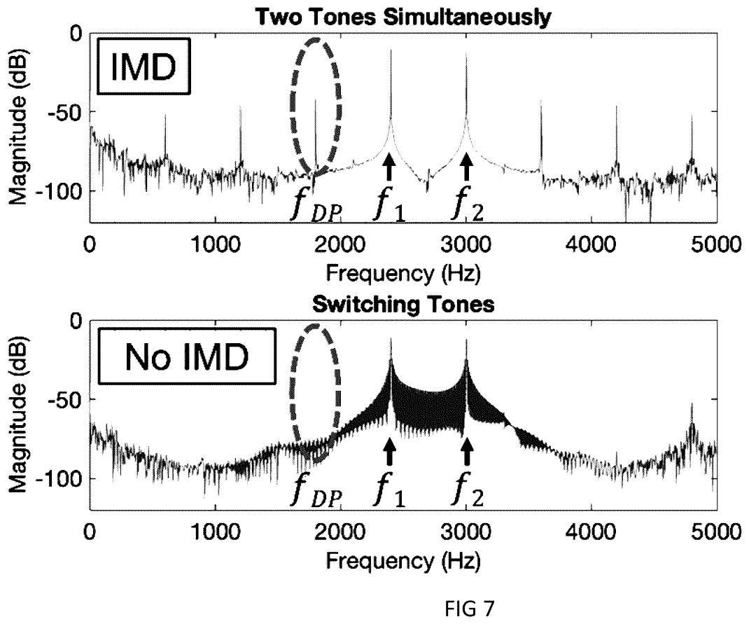

FIG. 7 shows another example of the subject matter described herein;

FIG. 8 shows another example of the subject matter described herein;

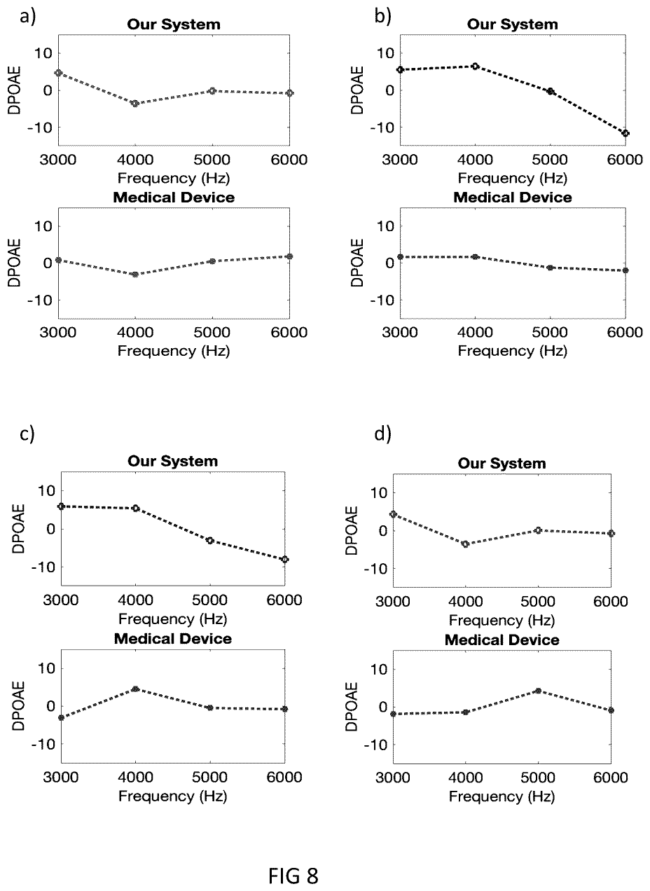

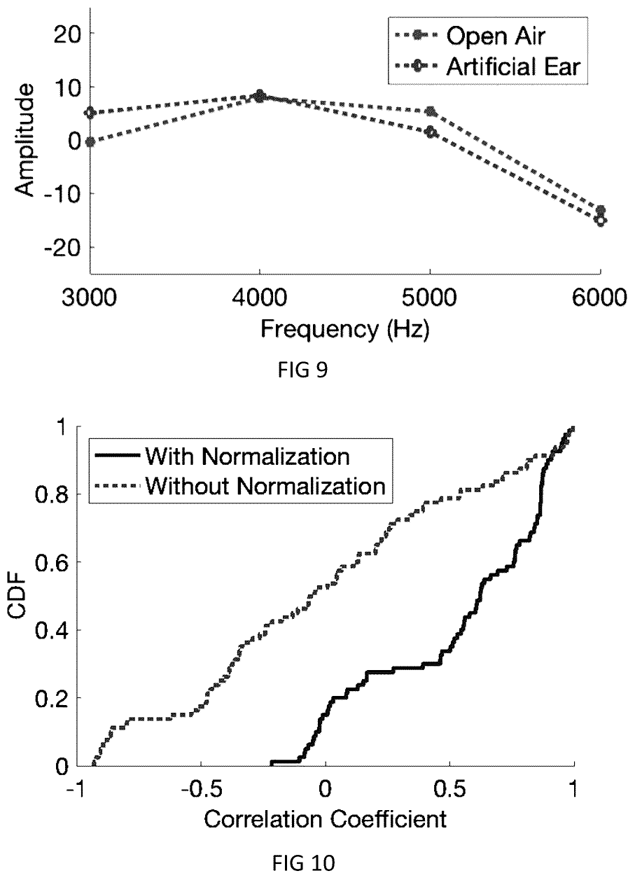

FIG. 9 shows another example of the subject matter described herein;

FIG. 10 shows another example of the subject matter described herein;

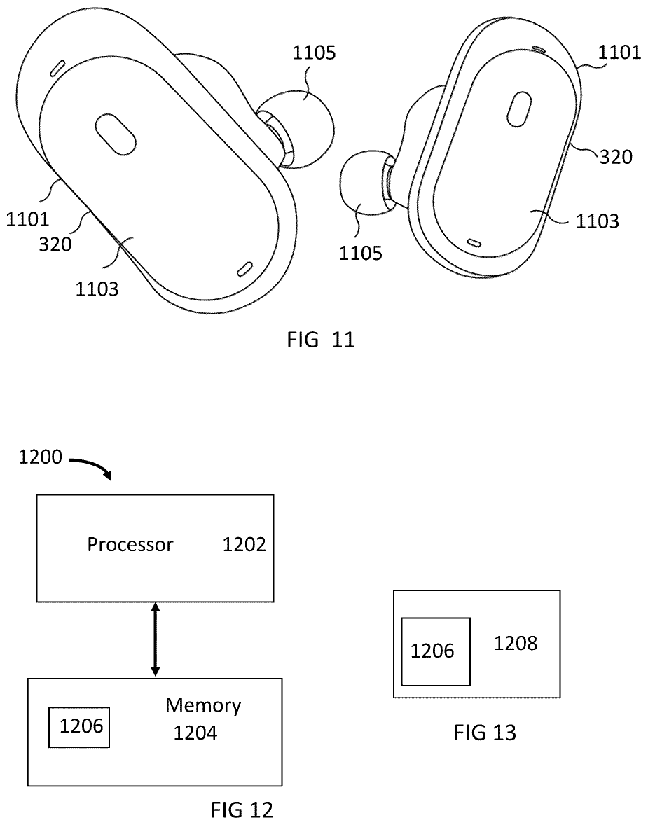

FIG. 11 shows another example of the subject matter described herein;

FIG. 12 shows another example of the subject matter described herein; and

FIG. 13 shows another example of the subject matter described herein.

The figures are not necessarily to scale. Certain features and views of the figures can be shown schematically or exaggerated in scale in the interest of clarity and conciseness. For example, the dimensions of some elements in the figures can be exaggerated relative to other elements to aid explication. Similar reference numerals are used in the figures to designate similar features. For clarity, all reference numerals are not necessarily displayed in all figures.

### DETAILED DESCRIPTION

Otoacoustic emissions (OAEs) are useful for evaluating the hearing level of a subject because their measurement does not require any active cooperation from the subject. Unlike other methods, such as audiometry, there is no need to obtain any feedback from a subject via a deliberate response. For example, there is no need for the subject to actuate a button or provide any feedback indicating how well they can hear.

In some examples, the subject is a human, and may be a human having their hearing evaluated. In some examples the subject may be any type of mammal. The lack of need to obtain a deliberate response from a subject is particularly useful for non-human mammals and human infants.

To measure OAEs otoacoustic signals are played into the ear of the subject and the response can be detected by one or more microphones positioned, for example in or close to the outer ear.

FIGS. 1A and 1B show example amplitude spectrums for the outputs of loudspeakers playing a pair of tones that can be used for measuring otoacoustic emissions.

FIG. 1A shows an amplitude spectrum for two loudspeakers playing the pair of tones. The amplitude spectrum is the combined output of the two loudspeakers. The x axis plots the frequency in Hz and the y axis plots the amplitude in dB. The audio signals being played comprise two frequency components or tones. The first frequency component is played by a first loudspeaker and the second frequency component is played by a second loudspeaker.

The first frequency component has a frequency f₁ of 1640 Hz and the second frequency component has a frequency f₂ of 2000 Hz. These two frequencies are examples of frequencies that could be used in audio signals for otoacoustic measurements. A plurality of different pairs of frequencies can be used to make the otoacoustic measurements.

The different frequency components can have different amplitudes. In this example the first frequency component has a larger amplitude than the second frequency component.

As shown in FIG. 1A there are no significant distortions when the pair of tones for otoacoustic measurements are played by two loudspeakers. There are no distortions with an amplitude above the general noise level.

FIG. 1B shows an amplitude spectrum for a single loudspeaker playing the pair of tones. The x axis plots the frequency in Hz and the y axis plots the amplitude in dB. The audio signals being played back comprise two frequency components or tones. The first frequency component has a frequency f₁ of 1640 Hz and the second frequency component has a frequency f₂ of 2000 Hz. These two frequencies are examples of frequencies that could be used in audio signals for otoacoustic measurements. A plurality of different pairs of frequencies would be used to make the otoacoustic measurements.

The different frequency components may have different amplitudes. In this example the first frequency component has a larger amplitude than the second frequency component.

In this example both the first frequency component and the second frequency component are played by the same loudspeaker.

As shown in FIG. 1B there are significant Intermodulation Distortions (IMDs). The IMDs arise due to the non-linearity of the loudspeaker. The IMDs arise due to the interaction of the respective frequency components with one another. The IMDs are at frequencies defined by the sums and differences of the first and second frequencies.

The example of FIG. 1B shows that there are multiple IMDs with an amplitude above the general noise level. In the example shown in FIG. 1B there are IMDs at 2f₁−f₂, 2f₁, 3f₁, 2f₁+f₂ that have an amplitude larger than the noise level.

The IMD at 2f₁−f₂ occurs at the same frequency as the cochlea response that is most commonly used for DPOAE measurements. This IMD caused by the non-linearity of the loudspeaker therefore prevents accurate OAE measurements being made because a microphone would detect both the IMD originating from the loudspeaker and also the OAE from the inner ear and would not necessarily be able to discriminate between the two. Even if attempts were made to discriminate between the loudspeaker-introduced IMD and the OAE, these would be dependent upon accurate calibration data and require special signal processing to extract the OAE from the combined frequency component. Other OAEs occur at other frequencies; however, these frequencies also correspond to IMD produced in the loudspeaker and similarly impede accurate OAE measurement.

The effect of IMDs can be particularly pronounced for devices such as earbuds which have to be small enough to fit wholly or partially into a subject's ear and so only have space for a small loudspeaker. The small loudspeakers have limitations on the movement of the cone and so can show higher non-linear behaviours than larger loudspeakers.

FIG. 2 shows an example method 200 according to examples of the disclosure. The method 200 could be implemented using any suitable apparatus or device. Example apparatuses that could be used to implement examples of the disclosure are shown below in FIGS. 3, 11 and 12.

The method 200 comprises, at block 202, causing alternating output of a first audio content and a second audio content, the first audio content comprising a first frequency and the second audio content comprising a different second frequency. The output of the first audio content and the output of the second audio content are entirely or substantially non-overlapping temporally. In some examples the output of the first audio content and the output of the second audio content being substantially non-overlapping temporally includes a small temporal overlap.

In some examples, the first audio content is a first pure tone, and the second audio content is a second pure tone. In other examples at least one of the first audio content and the second audio content comprise multiple frequencies. In some examples, the second frequency is between 1.15 and 1.3 times the first frequency, such as 1.22 times the first frequency.

In some examples causing alternating output of the first audio content and the second audio content comprises alternately outputting the first audio content but not the second audio content, and the second audio content but not the first audio content. In some, but not necessarily all, examples alternating output of the first audio content and the second audio content comprises alternately outputting substantially only the first pure tone and substantially only the second pure tone.

The first audio content and second audio content are configured to be played by a loudspeaker to enable OAEs within a subject's ear to be measured.

In some examples, causing alternating output of the first audio and the second audio content comprises causing output of the first audio content and the second audio content by a single loudspeaker to enable OAEs of a subject's ear to be measured.

The method 200 comprises, at block 204, receiving, responsive to output of the first audio content and the second audio content, an audio signal indicative of OAEs. In some examples, receiving an audio signal indicative of OAEs comprises receiving a microphone input indicative of distortion product OAEs.

In examples of this disclosure, for the purposes of DPOAE, a single loudspeaker is placed within a subject's ear and is used to alternately play a first frequency and a second frequency rather than the two frequencies being played simultaneously.

Within an ear the cochlear hairs are surrounded by a liquid. Based on fluid dynamic principles, liquids continue oscillating for a while even when the stimulus is removed. The liquid and the cochlear hairs will continue oscillating at the first frequency for a short period of time after output of the first frequency has ceased and so the cochlear hairs will oscillate at both the first and second frequency simultaneously and produce a response at a different third frequency. The third frequency is a combination tone based on the first and second frequencies. In practice, further combination tones will be produced at further frequencies.

Meanwhile, the cone of the loudspeaker has significantly less inertia and therefore stops oscillating at the first frequency substantially immediately once the output of the first frequency has ceased. Thus the loudspeaker does not oscillate at both the first and second frequency simultaneously and so does not produce IMDs. This would also be true of the microphone.

The absence of IMDs originating from the loudspeaker means that the microphone detects the OAEs at the third frequency without any competing nearby frequencies. This enables devices with a single loudspeaker to be used for DPOAE. This can be achieved without any need for signal processing, filtering, or noise cancellation. As such embodiments of the disclosure have the advantages of being more efficient, more accurate and having lower latency.

Additionally, devices with a single loudspeaker can be used for DPOAE without the need for calibration which makes the process faster and simpler.

Being able to perform DPOAE on devices with a single loudspeaker enables a much wider variety of devices to be used, including conventional earphones/headphones.

DPOAE using devices with a single loudspeaker have an advantage over devices with two loudspeakers as they avoid the issue of alignment of the two loudspeakers. Alignment issues can involve the two loudspeakers being offset from one another and so causing reflections of the outputting audio around the outer ear which can create additional IMDs.

Additionally, DPOAE using devices with a single loudspeaker allows the form factor of the device to be smaller. It can also avoid the need for a bulky separate device for the loudspeakers.

In some, but not necessarily all, examples alternating output of the first audio content and the second audio content comprises alternating output of the first audio content and the second audio content with temporal gaps between output of the first audio content and output of the second audio content of less than 100 ms, such as less than 20 ms or less than 5 ms. The gap needs to be short enough that the inertia in the cochlea results in the cochlear hairs oscillating at both the first and second frequency simultaneously and so producing a response at the different third frequency.

In some examples there is no temporal gap between output of the first audio content and output of the second audio content. In some examples there is a small temporal overlap between output of the first audio content and output of the second audio content. For example, the temporal overlap may be less than 5 ms, or less than 20 ms. A small temporal overlap can lead to a small IMD in the loudspeaker. As such IMD may be only reduced rather than eliminated.

In some examples, alternating output of the first audio content and the second audio content comprises alternating output of the first audio content and the second audio content with a changeover period of less than 100 ms, such as less than 20 ms or less than 5 ms. The changeover period is the period between outputting the first audio content at more than 80% of its greatest volume and outputting the second audio content at more than 80% of its greatest volume.

A smaller temporal gap or changeover period means that the cochlear hairs will be oscillating more strongly at the first frequency when they receive the second frequency. This leads to a higher amplitude cochlear response and so to more accurate DPOAE measurements.

In some examples, alternating output of the first audio content and the second audio content comprises alternating/switching output of the first audio content and the second audio content with a frequency of less than 50 Hz, such as less than 10 Hz. In some examples the output is alternated with a frequency of greater than 0.2 Hz, such as greater than 1 Hz, or between 1 to 10 Hz, such as approximately 4 Hz. The frequency of alternation can be chosen so as to reduce or minimize switching artefacts.

In some examples, alternating output of the first audio content and the second audio content comprises alternating output of the first audio content and the second audio content with a duty cycle of 20% to 80%, such as 40 to 60% or substantially 50%.

In some, but not necessarily all examples alternating output of the first audio content and the second audio content comprises alternating output of the first audio content and the second audio content with two or more repetitions of the first and second audio content. For example, with five or more repetitions.

FIG. 3 shows an example of an apparatus 300. The apparatus 300 could be used to implement methods such as the methods of FIG. 2, 4, or 5 or any other suitable methods or variations of the methods. The methods could be implemented using an apparatus as shown in FIGS. 11 and 12 or any other suitable type of apparatus or combinations of apparatus. An ear 50 of a subject is shown, including the ear canal 52, the inner ear 54 and the cochlea 56.

The illustrated apparatus 300 comprises two frequency generators 302, 304, a square wave generator 306, combiners 308, 310, 312, windowing functions 316, 318, a loudspeaker 325, a microphone 330 and an integrator 332. To create a first audio signal, the output of the first frequency generator 302 and the output of the square wave generator 306 are combined at a first combiner 308. To create a second audio signal, the output of the square wave generator 306 is inverted at an inverter 314 and then is combined with the output of the second frequency generator 304 at a second combiner 310.

The first and second audio signals may then be passed through windowing functions 316, 318. The first and second audio signals are combined at a third combiner 312 before being outputted by the loudspeaker 325. In this manner alternating output of the first audio content and the second audio content can be created, with the loudspeaker 325 switching from outputting the first audio content to outputting the second audio content and subsequently switching from outputting the second audio content to outputting the first audio content.

The first audio content and the second audio content travel down the ear canal 52 to the cochlea 56 where the cochlear hairs are stimulated. A cochlea response of OAE makes the reverse journey down the ear canal 52 and this audio signal is picked up by the microphone 330. The microphone input can then be transformed from the time domain to the frequency domain by an integrator 332. Based at least on the received audio signal, a property may be determined. The property may be a property related to the subject's ear 50.

The loudspeaker 325 could be a small loudspeaker 325 such as a loudspeaker 325 within an earbud 320 or another suitable device, for example a hearing aid. In the illustrated example the loudspeaker 325 and microphone 330 are located within the earbud 320. The other components may be located within the earbud 320 or in a separate device such as a smartphone, a computing device, or any other suitable device. The microphone 330 can comprise any means that can be configured to convert an acoustic input signal into a corresponding electrical output signal. The microphone 330 can be part of a digital signal processing device or any other suitable device.

It will be appreciated by those skilled in the art that in other examples the apparatus 300 may be different to that shown in FIG. 3 and that there are different ways of generating alternating audio content. For example, the apparatus 300 might not comprise a loudspeaker 325 or microphone 330, with these elements instead being part of a separate device. In some examples windowing functions 316, 318 might not be used. In some examples the apparatus 300 comprises a hearing aid. The apparatus 300 may comprise a combined hearing aid and headphone.

FIGS. 4 and 5 show example methods 400, 500 according to examples of the disclosure. The methods 400, 500 could be implemented using any suitable apparatus or device. Example apparatuses that could be used to implement examples of the disclosure are shown in FIGS. 3, 11 and 12. Blocks 202 and 204 are the same as is described with regard to FIG. 2.

The method 400 of FIG. 4 comprises, at block 406, determining, based at least on the received audio signal, the health of the subject's ear 50. Determining the health of the subject's ear 50 may comprise evaluating the hearing level of the subject's ear 50. For example, the cochlear hair response at a frequency can be used to determine the hearing level of a subject's ear 50 at the frequency. By repeating this procedure at different frequencies an audiometric sensitivity profile of a subject's ear 50 may be determined.

The method 500 of FIG. 5 comprises, at block 506, determining, based at least on the received audio signal, a configuration for outputting further audio content to the subject's ear 50.

In some examples the configuration for outputting further audio content comprises means for causing changes of at least one of amplitude and frequency of the audio content to be emitted. For example, if a subject's ear 50 has a poor response (e.g. a reduced amplitude of OAEs) at a certain frequency the volume may be increased for audio at that frequency. Additionally (or alternatively) audio at that frequency may be shifted to a frequency where the response from the subject's ear 50 is better.

In some examples determining the configuration comprises determining a plurality of frequencies configured to cause the subject to perceive a predetermined frequency as a psychoacoustic combination tone.

In some examples, the apparatus may store a log (or cause a log to be stored by another apparatus) that includes historical data indicating the health of the subject's ear 50. For example, the log might comprise results of DPOAE tests performed on the subject's ear 50 at different times. The data in the log may be compared to determine a change in the health of the subject's ear 50 over time, for example a reduction in OAEs in response to tones at particular frequencies, or at any frequencies. In response to a detected change in the subject's ear health, an alert informing of the change might be provided to the subject, to the subject's doctor, or to another person, organisation, or system.

The method 500 of FIG. 5 comprises, at block 508, causing output of the further audio content based at least in part on the configuration.

It will be apparent to those skilled in the art that the methods of FIGS. 4 and 5 may be combined, and that the same received audio signal may be used for both evaluating the health of the subject's ear 50 and determining a configuration for outputting further audio content.

It will also be apparent that the methods of FIGS. 2, 4 and 5 may be performed on each ear 50 of a subject independently. The methods may be performed on each ear 50 of the subject simultaneously without any need for input from the subject, making the procedure faster and less obtrusive to the subject. In some examples, the methods may be performed completely independently on each ear 50 of the subject. In other examples, some steps may be performed independently for each ear 50 whilst others are shared. For example, the same audio content may be outputted for each ear 50, whilst different microphone inputs are received for each ear 50.

The assessment of OAEs using simpler devices with a single loudspeaker 325, such as earbuds 320, provides opportunities for hearing screening in situations where access to medical equipment is limited.

In examples of the disclosure IMDs are reduced or eliminated in the signal captured by the microphone 330 as the pair of frequencies defined by the DPOAE are played sequentially rather than simultaneously. If we alternate between f₁ and f₂ of a pair of DPOAE frequencies, as shown in FIG. 6; then the ear 50 (in particular the liquid in the cochlea 56) still has memory of f₁ when f₂ is played, and vice versa. This would show a DPOAE signal at 2f₁−f₂ (OAE emission) for a period of time after the switching boundary.

FIG. 6 shows alternating/switching DPOAE playback for a first frequency f₁, and a second frequency f₂; (a) is a square window switching approach, (b) is a Hanning window switching approach, (c) is a zoomed-in region of (a) around the switching between f₁ and f₂, (d) is a zoomed-in region of (b) around the switching between f₁ and f₂. For the duration of τ/2 the loudspeaker 325 is playing f₁ and for the other τ/2 duration the loudspeaker 325 is playing f₂. In the illustrated example this pattern is repeated several times.

FIG. 7 shows results of a comparison of switching DPOAE (bottom), and simultaneously playing the two DPOAE tones (top) through a single loudspeaker 325. For these results an artificial ear without cochlear hairs was used. It is evident that switching DPOAE resolves the IMD problem present in simultaneous DPOAE tone playing, allowing DPOAE measurement in single loudspeaker earbuds 320.

FIG. 3 shows details of the proposed apparatus 300. It may be used for square-wave switching or windowed switching. For the pairs of DPOAE stimuli, one frequency will be present at the positive part of the switching control signal, and the second frequency will be present at the zero parts of the switching control signal. The switching period is governed by the parameter t which defines the duration of a complete switching cycle. Another control parameter from a switching control signal perspective, is the duty cycle γ. The duty cycle is the ratio of the positive cycle to the zero cycle. A 50% duty cycle would mean positive and zero have the same time width and so both frequencies are played for the same duration (i.e. γ=τ/2).

FIG. 6, shows examples of the pre-generated signal for a pair of DPOAE frequency pairs which can be played through the single loudspeaker 325 present in an off-the-shelf earbud 320. DPOAE analysis takes place on the signal captured by the internal facing microphone 330 of the earbud 320. After conversion from analogue to digital via an analog-to-digital converter, the signal can undergo a single point fast Fourier transform which is centered around the 2f₁−f₂ point. The OAE generated from the pairs of DPOAE frequencies will occur for a period of time β after every switching boundary. In some examples, β«γ.

Preliminary results were collected from a subject study that included 8 subjects (5-males and 3-females) spanning the age of 25 to 65 years. FIG. 8 shows a comparison of an example single loudspeaker apparatus 300 with alternating output of a first audio content and a second audio content against a medical device (GSI Corti) that is designed to measure DPOAE. The test sequence derives from the selection of DPOAE frequencies under consideration, specifically f₂ values of 3000, 4000, 5000, and 6000 Hz. The corresponding f₁ frequency is computed using f₂/1.22. These frequency pairs were alternated at a rate of 4 Hz (i.e., τ=0.25) for a duration of 6 seconds each, resulting in an approximate test sequence duration of 30 seconds (i.e., 4 (frequencies)*6 (secs per pair)+gaps in between). The switching method used was the square window as it produced the most reliable results. This test sequence was repeated ten times for each subject. The intensity level for f₁ was set to L₁=65 dB-SPL (sound pressure level), while f₂ was maintained at an intensity level of L₂=55 dB-SPL.

In FIG. 8, the top graphs, are the output of the single loudspeaker apparatus 300 (averaged over the 10 measurements), whereas the bottom graphs are the corresponding output for the same subject using the medical device (GSI Corti). It can be seen that there is a trend in the output of the single loudspeaker device 300 as compared to the medical device. This demonstrates that a single loudspeaker apparatus 300 with alternating output of a first audio content and a second audio content can trigger DPOAEs in subjects, hence enabling OAE measurement with a single speaker 325 without the IMD problem.

To confirm that the variations we are measuring are truly OAE-related, we conducted experiments both in open air and with an artificial ear. In these scenarios, where the cochlea 56 is absent, DPOAE generation is not expected. FIG. 9 demonstrates that both open-air and the artificial ear yield similar results. This confirms that all observed patterns in the experiments (as shown in FIG. 8) with different subjects indeed arise from DPOAEs triggered by the single loudspeaker apparatus 300.

To formally evaluate the performance of the single loudspeaker apparatus 300, we conducted a quantitative comparison with the medical device. We employed the correlation coefficient as the evaluation metric to measure the similarity between the DPOAE estimates generated by the single loudspeaker apparatus 300 and those acquired from the medical device. The higher correlation coefficient indicates higher similarity. FIG. 10 presents a Cumulative Distribution Function (CDF) plot of correlation coefficients between the single loudspeaker apparatus 300 and the medical device. It reveals that the single loudspeaker apparatus 300 achieves a median correlation coefficient of 0.65. Additionally, FIG. 10 highlights the significance of a normalization technique by demonstrating that, without normalization, the median correlation coefficient deteriorates to −0.48.

FIG. 11 shows example devices 1101 that could be used to measure otoacoustic emissions according to examples of the disclosure. The example devices 1101 comprise earbuds 320. The earbuds 320 comprise a housing 1103 and an inner ear portion 1105. The inner ear portion 1105 is sized and shaped to fit into the inner ear 54 of a subject. When the earbuds 320 are in use the inner ear portion is inserted into the inner ear 54 of the subject.

The housing 1103 can be configured to house an apparatus or any other suitable control means for controlling the devices 1101. An example apparatus 1200 is shown in FIG. 12.

FIG. 12 schematically illustrates an apparatus 1200 that can be used to implement examples of the disclosure. In some examples the apparatus 1200 is a controller and can be a chip or a chip-set. In some examples the apparatus 1200 is, or is provided within, any suitable device such as earbuds 320 or a device such as a smartphone that can be configured to communicate with the earbuds 320. The apparatus 1200 of FIG. 12 may be the apparatus 300 of FIG. 3 and may comprise some or all of the features described regarding the apparatus 300 of FIG. 3.

FIG. 12 illustrates an example of a controller 1200. Implementation of a controller 1200 may be as controller circuitry. The controller 1200 may be implemented in hardware alone, have certain aspects in software including firmware alone or can be a combination of hardware and software (including firmware).

As illustrated in FIG. 12 the controller 1200 may be implemented using instructions that enable hardware functionality, for example, by using executable instructions 1206 in a general-purpose or special-purpose processor 1202 that may be stored on a machine readable storage medium (disk, memory etc.) to be executed by such a processor 1202.

The processor 1202 is configured to read from and write to the memory 1204. The processor 1202 may also comprise an output interface via which data and/or commands are output by the processor 1202 and an input interface via which data and/or commands are input to the processor 1202.

The memory 1204 stores instructions, program, or code 1206 that controls the operation of the apparatus 1200 when loaded into the processor 1202. The computer program instructions, program or code am 1206, provide the logic and routines that enables the apparatus 1200 to perform the methods illustrated in the accompanying FIGs. The processor 1202 by reading the memory 1204 is configured to load and execute the instructions, program, or code 1206.

The apparatus 1200 comprises:
at least one processor 1202; and
at least one memory 1204 storing instructions that, when executed by the at least one processor 1202, cause the apparatus at least to:
   cause alternating output of a first audio content and a second audio content, the first audio content comprising a first frequency and the second audio content comprising a different second frequency;
   receive, responsive to output of the first audio content and the second audio content, an audio signal indicative of otoacoustic emissions.

As illustrated in FIG. 13, the instructions, program, or code 1206 may arrive at the apparatus 1200 via any suitable delivery mechanism 1208. The delivery mechanism 1208 may be, for example, a machine readable medium, a computer-readable medium, a non-transitory computer-readable storage medium, a computer program product, a memory device, a record medium such as a Compact Disc Read-Only Memory (CD-ROM) or a Digital Versatile Disc (DVD) or a solid-state memory, an article of manufacture that comprises or tangibly embodies the computer program 1206. The delivery mechanism may be a signal configured to reliably transfer the computer program 1206. The apparatus 1200 may propagate or transmit the computer program 1206 as a computer data signal.

The term “non-transitory” as used herein, is a limitation of the medium itself (i.e., tangible, not a signal) as opposed to a limitation on data storage persistency (for example, RAM vs. ROM).

Computer program instructions for causing an apparatus to perform at least the following or for performing at least the following:
causing alternating output of a first audio content and a second audio content, the first audio content comprising a first frequency and the second audio content comprising a different second frequency;
receiving, responsive to output of the first audio content and the second audio content, an audio signal indicative of otoacoustic emissions.

The computer program instructions may be comprised in a computer program, a non-transitory computer readable medium, a computer program product, a machine readable medium. In some but not necessarily all examples, the computer program instructions may be distributed over more than one computer program.

Although the memory 1204 is illustrated as a single component/circuitry it may be implemented as one or more separate components/circuitry some or all of which may be integrated/removable and/or may provide permanent/semi-permanent/dynamic/cached storage.

Although the processor 1202 is illustrated as a single component/circuitry it may be implemented as one or more separate components/circuitry some or all of which may be integrated/removable. The processor 1202 may be a single core or multi-core processor.

References to ‘computer-readable storage medium’, ‘computer program product’, ‘tangibly embodied computer program’ etc. or a ‘controller’, ‘computer’, ‘processor’ etc. should be understood to encompass not only computers having different architectures such as single/multi-processor architectures and sequential (Von Neumann)/parallel architectures but also specialized circuits such as field-programmable gate arrays (FPGA), application specific circuits (ASIC), signal processing devices and other processing circuitry. References to computer program, instructions, code etc. should be understood to encompass software for a programmable processor or firmware such as, for example, the programmable content of a hardware device whether instructions for a processor, or configuration settings for a fixed-function device, gate array or programmable logic device etc.

As used in this application, the term ‘circuitry’ may refer to one or more or all the following:
(a) hardware-only circuitry implementations (such as implementations in only analog and/or digital circuitry) and
(b) combinations of hardware circuits and software, such as (as applicable):
   i. a combination of analog and/or digital hardware circuit(s) with software/firmware and
   ii. any portions of hardware processor(s) with software (including digital signal processor(s)), software, and memory or memories that work together to cause an apparatus, such as a mobile phone or server, to perform various functions and
(c) hardware circuit(s) and or processor(s), such as a microprocessor(s) or a portion of a microprocessor(s), that requires software (for example, firmware) for operation, but the software may not be present when it is not needed for operation.

This definition of circuitry applies to all uses of this term in this application, including in any claims. As a further example, as used in this application, the term circuitry also covers an implementation of merely a hardware circuit or processor and its (or their) accompanying software and/or firmware. The term circuitry also covers, for example and if applicable to the claim element, a baseband integrated circuit for a mobile device or a similar integrated circuit in a server, a cellular network device, or other computing or network device.

The blocks illustrated in the accompanying Figs may represent steps in a method and/or sections of code in the computer program 1206. The illustration of a particular order to the blocks does not necessarily imply that there is a required or preferred order for the blocks and the order and arrangement of the block may be varied. Furthermore, it may be possible for some blocks to be omitted.

As used here ‘module’ refers to a unit or apparatus that excludes certain parts/components that would be added by an end manufacturer or a subject. The apparatus 1200 can, for example be a module. A controller 1200 of the apparatus 1200 can, for example be a module.

Where a structural feature has been described, it may be replaced by means for performing one or more of the functions of the structural feature whether that function or those functions are explicitly or implicitly described.

The apparatus can be provided in an electronic device, for example, a mobile terminal, according to an example of the present disclosure. It should be understood, however, that a mobile terminal is merely illustrative of an electronic device that would benefit from examples of implementations of the present disclosure and, therefore, should not be taken to limit the scope of the present disclosure to the same. While in certain implementation examples, the apparatus can be provided in a mobile terminal, other types of electronic devices, such as, but not limited to: mobile communication devices, hand portable electronic devices, wearable computing devices, portable digital assistants (PDAs), pagers, mobile computers, desktop computers, televisions, gaming devices, laptop computers, cameras, video recorders, GPS devices and other types of electronic systems, can readily employ examples of the present disclosure. Furthermore, devices can readily employ examples of the present disclosure regardless of their intent to provide mobility.

The term ‘comprise’ is used in this document with an inclusive not an exclusive meaning. That is any reference to X comprising Y indicates that X may comprise only one Y or may comprise more than one Y. If it is intended to use ‘comprise’ with an exclusive meaning then it will be made clear in the context by referring to ‘comprising only one . . . ’ or by using ‘consisting.’

In this description, the wording ‘connect’, ‘couple’ and ‘communication’ and their derivatives mean operationally connected/coupled/in communication. It should be appreciated that any number or combination of intervening components can exist (including no intervening components), i.e., to provide direct or indirect connection/coupling/communication. Any such intervening components can include hardware and/or software components.

As used herein, the term “determine/determining” (and grammatical variants thereof) can include, not least: calculating, computing, processing, deriving, measuring, investigating, identifying, looking up (for example, looking up in a table, a database, or another data structure), ascertaining and the like. Also, “determining” can include receiving (for example, receiving information), accessing (for example, accessing data in a memory), obtaining and the like. Also, “determine/determining” can include resolving, selecting, choosing, establishing, and the like.

In this description, reference has been made to various examples. The description of features or functions in relation to an example indicates that those features or functions are present in that example. The use of the term ‘example’ or ‘for example’ or ‘can’ or ‘may’ in the text denotes, whether explicitly stated or not, that such features or functions are present in at least the described example, whether described as an example or not, and that they can be, but are not necessarily, present in some of or all other examples. Thus ‘example’, ‘for example’, ‘can’, or ‘may’ refers to a particular instance in a class of examples. A property of the instance can be a property of only that instance or a property of the class or a property of a sub-class of the class that includes some but not all the instances in the class. It is therefore implicitly disclosed that a feature described with reference to one example but not with reference to another example, can where possible be used in that other example as part of a working combination but does not necessarily have to be used in that other example.

As used herein, “at least one of the following:” and “at least one of” and similar wording, where the list of two or more elements are joined by “and” or “or” mean at least any one of the elements, or at least any two or more of the elements, or at least all the elements.

Although examples have been described in the preceding paragraphs with reference to various examples, it should be appreciated that modifications to the examples given can be made without departing from the scope of the claims.

Features described in the preceding description may be used in combinations other than the combinations explicitly described above.

Although functions have been described with reference to certain features, those functions may be performable by other features whether described or not.

The description of a feature, such as an apparatus or a component of an apparatus, configured to perform a function, or for performing a function, should additionally be considered to also disclose a method of performing that function. For example, description of an apparatus configured to perform one or more actions, or for performing one or more actions, should additionally be considered to disclose a method of performing those one or more actions with or without the apparatus.

Although features have been described with reference to certain examples, those features may also be present in other examples whether described or not.

The term ‘a’, ‘an’ or ‘the’ is used in this document with an inclusive not an exclusive meaning. That is any reference to X comprising a/an/the Y indicates that X may comprise only one Y or may comprise more than one Y unless the context clearly indicates the contrary. If it is intended to use ‘a’, ‘an’ or ‘the’ with an exclusive meaning then it will be made clear in the context. In some circumstances the use of ‘at least one’ or ‘one or more’ may be used to emphasis an inclusive meaning but the absence of these terms should not be taken to infer any exclusive meaning.

The presence of a feature (or combination of features) in a claim is a reference to that feature or (combination of features) itself and to features that achieve substantially the same technical effect (equivalent features). The equivalent features include, for example, features that are variants and achieve substantially the same result in substantially the same way. The equivalent features include, for example, features that perform substantially the same function, in substantially the same way to achieve substantially the same result.

In this description, reference has been made to various examples using adjectives or adjectival phrases to describe characteristics of the examples. Such a description of a characteristic in relation to an example indicates that the characteristic is present in some examples exactly as described and is present in other examples substantially as described.

The above description describes some examples of the present disclosure however those of ordinary skill in the art will be aware of possible alternative structures and method features which offer equivalent functionality to the specific examples of such structures and features described herein above and which for the sake of brevity and clarity have been omitted from the above description. Nonetheless, the above description should be read as implicitly including reference to such alternative structures and method features which provide equivalent functionality unless such alternative structures or method features are explicitly excluded in the above description of the examples of the present disclosure.

Whilst endeavoring in the foregoing specification to draw attention to those features believed to be of importance the Applicant may seek protection via the claims in respect of any patentable feature or combination of features hereinbefore referred to and/or shown in the drawings whether or not emphasis has been placed thereon.

1-15. (canceled)

16. An apparatus comprising:
at least one processor; and
at least one memory storing instructions that, when executed by the at least one processor, cause the apparatus at least to:
cause alternating output of a first audio content and a second audio content, the first audio content comprising a first frequency and the second audio content comprising a different second frequency; and
receive, responsive to output of the first audio content and the second audio content, an audio signal indicative of otoacoustic emissions.

17. The apparatus of claim 16, wherein the output of the first audio content and the output of the second audio content are non-overlapping temporally.

18. The apparatus of claim 16, wherein the first audio content is a first pure tone and the second audio content is a second pure tone.

19. The apparatus of claim 16, wherein the second frequency is 1.15 to 1.3 times the first frequency.

20. The apparatus of claim 16, wherein causing alternating output of the first audio content and the second audio content comprises alternating output of the first audio content and the second audio content with gaps between output of the first audio content and output of the second audio content of less than 100 ms.

21. The apparatus of claim 16, wherein receiving an audio signal indicative of otoacoustic emissions comprises receiving a microphone input indicative of distortion product otoacoustic emissions.

22. The apparatus of claim 16, wherein causing alternating output of the first audio content and the second audio content comprises, causing output of the first audio content and the second audio content by a single loudspeaker to enable otoacoustic emissions within a subject's ear to be measured.

23. The apparatus of claim 16, wherein causing alternating output of the first audio content and the second audio content comprises causing output of the first audio content and the second audio content to enable otoacoustic emissions within a subject's ear to be measured, and wherein the instructions, when executed by the at least one processor, further cause the apparatus at least to determine, based at least on the received audio signal, the health of the subject's ear.

24. The apparatus of claim 16, wherein causing alternating output of the first audio content and the second audio content comprises causing output of the first audio content and the second audio content to enable otoacoustic emissions within a subject's ear to be measured, and wherein the instructions, when executed by the at least one processor, further cause the apparatus at least to determine, based at least on the received audio signal, a configuration for outputting further audio content to the subject's ear.

25. The apparatus of claim 24, wherein the instructions, when executed by the at least one processor, further cause the apparatus at least to cause output of the further audio content based at least in part on the configuration.

26. A method comprising,
causing alternating output of a first audio content and a second audio content, the first audio content comprising a first frequency and the second audio content comprising a different second frequency; and
receiving, responsive to output of the first audio content and the second audio content, an audio signal indicative of otoacoustic emissions.

27. The method of claim 26, wherein the first audio content is a first pure tone and the second audio content is a second pure tone.

28. The method of claim 26, wherein causing alternating output of the first audio content and the second audio content comprises, causing output of the first audio content and the second audio content by a single loudspeaker to enable otoacoustic emissions within a subject's ear to be measured.

29. A non-transitory computer readable medium comprising program instructions stored thereon for performing at least the following:
causing alternating output of a first audio content and a second audio content, the first audio content comprising a first frequency and the second audio content comprising a different second frequency; and
receiving, responsive to output of the first audio content and the second audio content, an audio signal indicative of otoacoustic emissions.

30. The non-transitory computer readable medium of claim 29, wherein the first audio content is a first pure tone and the second audio content is a second pure tone.
